(19) United States
(12) Patent Application Publication
(10) Pub. No.: US 2023/0405281 A1
(43) Pub. Date: Dec. 21, 2023

(54) OVER-THE-NEEDLE INTRAVENOUS INTRODUCER ASSEMBLY

(71) Applicant: TUCANN MEDICAL PTY LTD, Berwick, Victoria (AU)

(72) Inventors: Maryam SOOMRO, Berwick, Victoria (AU); Kenneth Wei Hung WONG, Berwick, Victoria (AU); Santiago Andres BELTRAN DIAZ, Berwick, Victoria (AU); Brody PAYNE, Berwick, Victoria (AU); Fay GIBSON, Berwick, Victoria (AU)

(73) Assignee: TUCANN MEDICAL PTY LTD, Berwick, Victoria (AU)

(21) Appl. No.: 18/265,115

(22) PCT Filed: Dec. 3, 2021
(86) PCT No.: PCT/IB2021/061273

§ 371 Date: Jun. 2, 2023

(30) Foreign Application Priority Data
Dec. 4, 2020 (AU) 2020904495

(51) Int. Cl. A61M 25/06; A61M 39/10; A61M 39/22

(52) U.S. Cl. CPC A61M 25/0606; A61M 39/10; A61M 25/0631; A61M 25/0693; A61M 39/22

(57) ABSTRACT

An assembly, comprising a catheter hub defining a hub cavity extending axially between proximal and distal ends of the catheter hub, a female luer connector extending from within the proximal end of the catheter hub, a catheter tube extending from within the distal end of the catheter hub and a pinch valve body axially movable in the hub cavity and defining a valve conduit that fluidly connects the female luer connector and the catheter tube, wherein the pinch valve body has a distal end sheathed over the catheter tube, and a proximal end comprising proximal and distal pinch valves, wherein the female luer connector has internal pinch surfaces configured so that axial movement of the pinch valve body in the hub cavity positively opens and closes the proximal and distal pinch valves in predetermined succession or order as either a needle is proximally withdrawn from the catheter tube, or a male luer connector is distally inserted into, or proximally withdrawn from, the female luer connector.

### FIELD

The present invention relates generally to an over-the-needle intravenous introducer assembly for cannulas or catheters.

### BACKGROUND

Over-the-needle intravenous introducer assemblies for introducing cannulas or catheters into a patient's vein generally comprise an over-the-needle catheter hub having a catheter tube at a distal end, and a luer connector at a proximal end for administering fluids.

Existing over-the-needle intravenous introducer assemblies suffer several shortcomings. These include accidental needlestick injury to medical personnel, and contamination of medical personnel from backflow of the patient's blood. Existing assemblies are also difficult to insert into patients while maintaining aseptic non-touch technique, increasing the risk of poor insertion technique by contacting key sites of infections due to human error, and thus higher chances of patient infection. The difficulty in adhering to aseptic non-touch technique also puts the patient at risk of infection by transferring the bacteria from the administering medical personnel to the patient.

Over-the-needle intravenous introducer assemblies for cannulas or catheters have been developed to reduce the risks of needlestick injury and blood contamination, but such assemblies are complicated and require additional components, steps and time to introduce the cannula or catheter in the patient's vein. Such assemblies are particularly ill-suited for use in military and emergency medical settings, including pre-hospital (or battlefield) and in-hospital emergency medical settings.

In view of this background, there is an unmet need for improved over-the-needle intravenous introducer assemblies for cannulas or catheters.

### SUMMARY

According to the present invention, there is provided an assembly, comprising:
a catheter hub defining a hub cavity extending axially between proximal and distal ends of the catheter hub;
a female luer connector extending from within the proximal end of the catheter hub;
a catheter tube extending from within the distal end of the catheter hub; and
a pinch valve body axially movable in the hub cavity and defining a valve conduit that fluidly connects the female luer connector and the catheter tube;
wherein the pinch valve body has a distal end sheathed over the catheter tube, and a proximal end comprising proximal and distal pinch valves; and
wherein the female luer connector has internal pinch surfaces configured so that axial movement of the pinch valve body in the hub cavity positively opens and closes the proximal and distal pinch valves in predetermined succession or order as either a needle is proximally withdrawn from the catheter tube, or a male luer connector is distally inserted into, or proximally withdrawn from, the female luer connector.

The proximal and distal pinch valves may comprise a luer receiving valve and an internal shut-off valve respectively.

The proximal and distal pinch valves may be closed initially, and a needle may be pre-inserted in the catheter hub with a proximal portion in the valve conduit of the pinch valve body and a distal portion in the catheter tube.

Partial proximal withdrawal of the needle from the catheter hub may open the distal and proximal pinch valves in succession.

Full proximal withdrawal of the needle from the catheter hub may close the distal and proximal pinch valves in succession.

Distal insertion of the male luer connector into the female luer connector of the catheter hub may open the proximal and distal pinch valves in succession. This may create a sealed fluid path between the proximal end of the catheter hub and the catheter tube on the distal end of the catheter hub.

Proximal withdrawal of the male luer connector from the female luer connector of the catheter hub may close the distal and proximal pinch valves in succession.

The pinch valve body may be biased distally within the hub cavity to positively close the proximal and distal pinch valves.

The catheter hub may have an inwardly projecting tube portion at the distal end of the catheter hub, wherein the catheter tube extends distally from within the inwardly projecting tube portion, and the pinch valve body is axially movable on the inwardly projecting tube portion.

The pinch valve body may be biased distally within the hub cavity by a spring sheathed on the inwardly projecting tube portion.

Alternatively, elastomeric projections may be provided at the distal end of the valve body, wherein the elastomeric projections are sheathed over the inwardly projecting tube portion of the hub cavity to distally bias the pinch valve body.

The assembly may further comprise a manual needle withdrawal device removably connected to the female luer connector of the catheter hub, wherein the manual needle withdrawal device has a low form factor and is configured so that disconnection from the catheter hub manually distally withdraws the needle from the catheter hub and automatically applies a safety clip to the distal tip of the needle when fully distally withdrawn from the catheter hub.

The manual needle withdrawal device may comprise a flashback chamber configured to allow visualisation of an initial flow of blood upon insertion of a distal tip of the needle into a vein.

Alternatively, the assembly may further comprise an automatic needle withdrawal device removably connected to the female luer connector of the catheter hub, wherein the automatic needle withdrawal device comprises:
a flashback chamber configured to allow visualisation of an initial flow of blood upon insertion of a distal tip of the needle into a vein;
a needle enclosure configured to enclose the needle when fully distally withdrawn from the catheter hub;
a needle withdrawal mechanism configured to be triggered to automatically distally withdraw the needle from the catheter hub and move the needle to the needle enclosure when fully distally withdrawn from the catheter hub; and
a trigger mechanism configured to:
   trigger the needle withdrawal mechanism to fully proximally withdraw the needle into the needle enclosure, and disconnect the automatic needle withdrawal device from the catheter hub after the needle is fully proximally withdrawn into the needle enclosure; and
   before the needle is fully proximally withdrawn into the needle enclosure, prevent either:
      the needle being withdrawn inadvertently or prematurely; or
      the automatic needle withdrawal device being disconnected from the catheter hub inadvertently or prematurely.

The needle withdrawal mechanism may comprise a spring-loaded plunger arranged inside the needle enclosure, wherein the spring-loaded plunger is configured to mount a proximal end of the needle, and is spring-biased to proximally withdraw the needle into the needle enclosure.

The trigger mechanism may comprise a leaf spring arm integrally formed on a distal end of the automatic needle withdrawal device, wherein the leaf spring arm is configured to be activated automatically upon proximal withdrawal of the needle enclosure from the catheter hub to trigger automatic proximal withdrawal of the needle.

Alternatively, the trigger mechanism may comprise a non-integrally formed spring-loaded reciprocating arm arranged between a distal end of the automatic needle withdrawal device and the proximal end of the catheter hub, wherein the spring-loaded reciprocating arm is configured to be activated automatically upon proximal withdrawal of the needle enclosure from the catheter hub to trigger automatic proximal withdrawal of the needle.

The present invention also provides a method, comprising:
providing a needle inside a catheter tube at a distal end of a catheter hub having a female luer connector at a proximal end, wherein the catheter tube and female luer connector are fluidly connected by a pinch valve body that is proximally movable inside a hub cavity of the catheter hub, and wherein the pinch valve body has a distal end sheathed over the catheter tube, and a proximal end comprising proximal and distal pinch valves that are that are configured to be closed in succession by internal pinch surfaces of the female catheter connector upon proximal movement of the pinch valve body;
proximally withdrawing the needle from the catheter tube to proximally move the pinch valve body;
closing the proximal and distal pinch valves in succession upon full proximal withdrawal of the needle from the catheter hub.

The method may facilitate adherence to aseptic non-touch technique during introduction of a catheter or cannula into a vein of a patient.

### BRIEF DESCRIPTION OF DRAWINGS

Embodiments of the invention will now be described by way of example only with reference to the accompanying drawings, in which:

FIGS. 1 to 4 are cross-section side views showing sequential operation states of an over-the-needle intravenous introducer assembly for cannulas or catheters according to one embodiment of the present invention;

FIGS. 5 to 8 are cross-section side views showing sequential operation states of another embodiment of the invention; and

FIGS. 9 to 12 are cross-section side views showing sequential operation states of a further embodiment of the invention.

### DESCRIPTION OF EMBODIMENTS

Referring to the drawings, an over-the-needle intravenous introducer assembly 10 for cannulas or catheters according to an embodiment of the present invention may generally comprise a catheter hub 12 defining a hub cavity 14 extending axially between proximal and distal ends of the catheter hub 12, and a female luer connector 16 extending from within the proximal end of the catheter hub 12.

A catheter tube 18 may extend from within the distal end of the catheter hub 12. A pinch valve body 20 may be axially movable in the hub cavity 14. The pinch valve body may define a valve conduit 22 that fluidly connects the female luer connector 16 and the catheter tube 18.

The pinch valve body 20 may have a distal end sheathed over the catheter tube 18, and a proximal end comprising proximal and distal pinch valves 24, 26. The proximal and distal pinch valves 24, 26 may comprise a luer receiving valve 24 and an internal shut-off valve 26 respectively.

The female luer connector 16 may have internal pinch surfaces 28 configured so that axial movement of the pinch valve body 20 in the hub cavity 14 positively opens and closes the proximal and distal pinch valves 24, 26 in predetermined succession or order as either a needle 30 is proximally withdrawn from the catheter tube 18, or a male luer connector (not shown) is distally inserted into, or proximally withdrawn from, the female luer connector 16. As the male luer connector is inserted into the female luer connector 16, a sealed fluid pathway is created to enable access to the patient's vein.

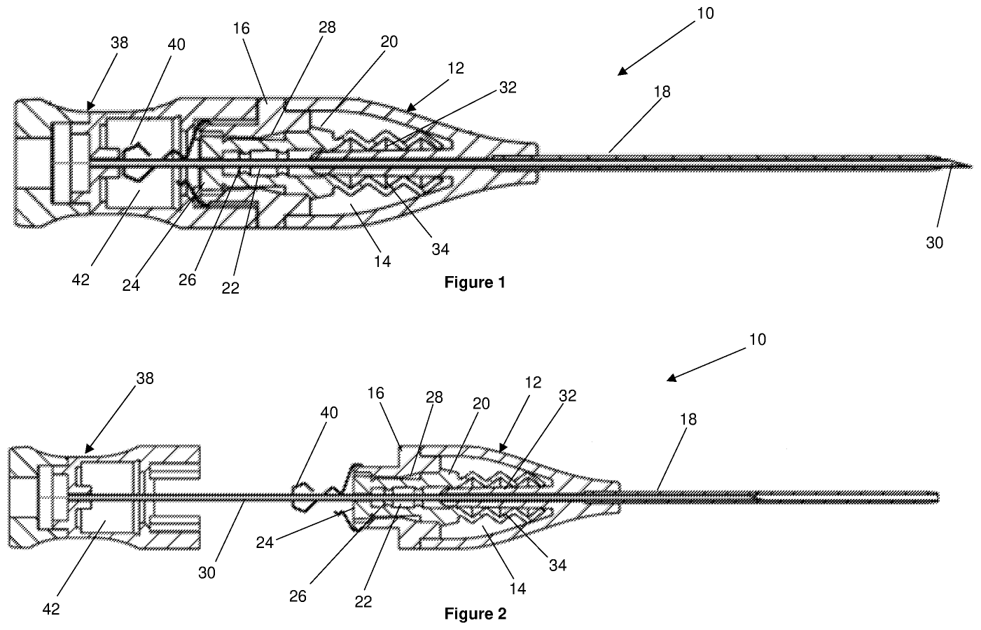

FIG. 1 shows the assembly 10 in an initial ‘ready-to-use’ operational state. In this state, the proximal and distal pinch valves 24, 26 may be closed initially, and the needle 30 may be pre-inserted in the catheter hub 12 with a proximal portion in the valve conduit 22 of the pinch valve body 20 and a distal portion in the catheter tube 18. In this operational state, the assembly 10 may be used for over-the-needle introduction of the catheter tube into a patient's vein.

FIG. 2 shows the assembly 10 in a successive operational state with the needle partially withdrawn from the catheter hub 12. In this operational state, partial proximal withdrawal of the needle 30 from the catheter hub 12 may open the distal and proximal pinch valves 26, 24 in succession.

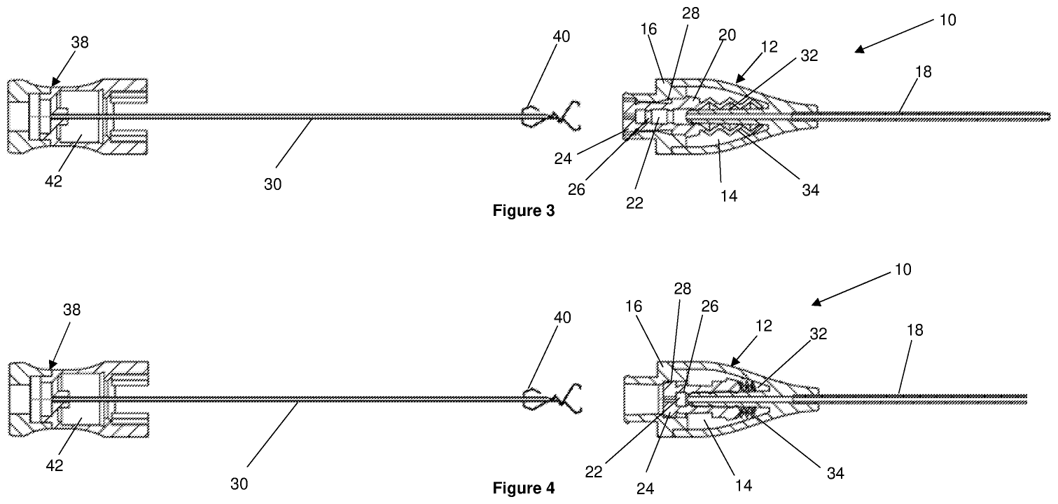

FIG. 3 shows the assembly 10 in a successive operational state with the needle fully withdrawn from the catheter hub 12. In this operational state, full proximal withdrawal of the needle 30 from the catheter hub 12 may close the distal and proximal pinch valves 26, 24 in succession. The distal pinch valve 26 may be first to close to act as an internal shut-off valve to prevent a flow of blood from the female luer connector 16 upon full withdrawal of the needle 30 from the catheter hub 12. The proximal pinch valve 24 may then close in succession to seal the female luer connector 16.

FIG. 4 shows the assembly 10 in a successive operational state with the male luer connector (not shown) inserted in the female luer connector 16 of the catheter hub 12. In this operational state, distal insertion of the male luer connector (not shown) into the female luer connector 16 of the catheter hub 12 may open the proximal and distal pinch valves 24, 26 in succession. The distal pinch valve 26 may be first to open to act as a luer receiving valve upon insertion of the male luer connector into the female luer connector 16. The distal pinch valve 26 may then open in succession to fluidly connect the male luer connector to the catheter tube 18 to administer fluid to the patient's vein.

Conversely, proximal withdrawal of the male luer connector from the female luer connector 16 of the catheter hub 12 may close the distal and proximal pinch valves 26, 24 in succession. Again, the distal pinch valve 26 may be first to close to act as an internal shut-off valve to prevent a flow of fluid from the female luer connector 16 upon full withdrawal of the needle 30 from the catheter hub 12. The proximal pinch valve 24 may then close in succession to seal the female luer connector 16.

The pinch valve body 20 may be biased distally within the hub cavity 14 to positively close the proximal and distal pinch valves 24, 26 when the assembly 10 is in a passive state after use for needle 30 withdrawal and fluid administration. The catheter hub 12 may have an inwardly projecting tube portion 32 at the distal end of the catheter hub 12. The catheter tube 18 may extend distally from within the inwardly projecting tube portion 32, and the pinch valve body 20 may be axially movable on the inwardly projecting tube portion 32.

In the embodiment shown in FIGS. 1 to 4, elastomeric projections 34 may be provided at the distal end of the pinch valve body 20. The elastomeric projections 34 may be sheathed over the inwardly projecting tube portion 32 of the hub cavity 14 to distally bias the pinch valve body 20.

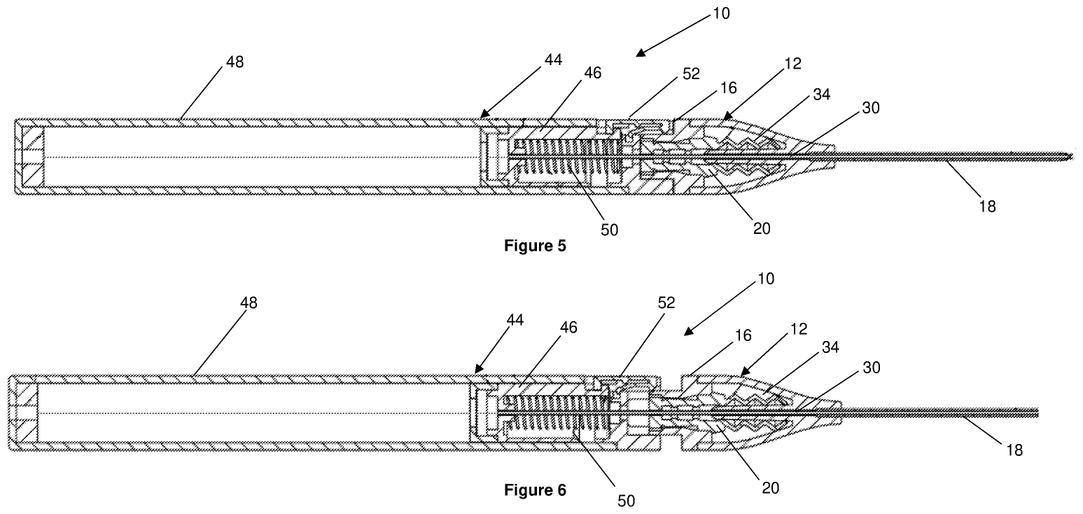

In the alternative embodiments shown in FIGS. 5 to 12, the pinch valve body 20 may be biased distally within the hub cavity 14 by a spring 36 sheathed on the inwardly projecting tube portion 32.

In the embodiment shown in FIGS. 1 to 4, the assembly 10 may further comprise a manual needle withdrawal device 38 removably connected to the female luer connector 16 of the catheter hub 12. The manual needle withdrawal device 38 may have a low form factor, and may be configured so that disconnection from the catheter hub 12 manually distally withdraws the needle 30 from the catheter hub 12 and automatically applies a safety clip 40 to the distal tip of the needle 30 when fully distally withdrawn from the catheter hub 12.

The safety clip 40 may, for example, comprise a spring clip that springs into position to block the distal tip of the needle 30 when fully proximally withdrawn from the catheter hub 12 to thereby facilitate safe handling of the needle 30.

The manual needle withdrawal device 38 may further comprise a flashback chamber 42 configured to allow visualisation of an initial flow of blood upon insertion of a distal tip of the needle 30 into a patient's vein.

FIGS. 1 to 4 show an embodiment of the assembly 10 in each of four successive operational states that may be referred to, respectively, as:
a first state ‘ready-to-use’ for needle insertion (FIG. 1);
a second state with partial needle withdrawal (FIG. 2);
a third state with full needle withdrawal (FIG. 3); and
a fourth state ‘ready-to-use’ for intravenous fluid administration (FIG. 4).

In alternative embodiments shown in FIGS. 5 to 16, the assembly 10 may further comprise an automatic needle withdrawal device 44 removably connected to the female luer connector 16 of the catheter hub 12. The automatic needle withdrawal device 44 may comprise a flashback chamber 46 configured to allow visualisation of an initial flow of blood upon insertion of a distal tip of the needle 30 into a patient's vein. A needle enclosure 48 may be connected to a distal end of the flashback chamber 46, and may be configured to enclose the needle 30 when fully distally withdrawn from the catheter hub 12.

The automatic needle withdrawal device 44 may further comprise a needle withdrawal mechanism 50 and a trigger mechanism 52. The needle withdrawal mechanism 50 may be configured to be triggered to automatically distally withdraw the needle 30 from the catheter hub 12, and move the needle 12 to a position fully inside the needle enclosure 48 when fully distally withdrawn from the catheter hub 12.

The trigger mechanism 52 may be configured to trigger the needle withdrawal mechanism 50 to automatically fully proximally withdraw the needle 30 into the needle enclosure 48, and to automatically disconnect the automatic needle withdrawal device 44 from the catheter hub 12 after the needle 30 is fully proximally withdraw the needle into the needle enclosure 48.

The trigger mechanism 52 may be further configured, before the needle 30 is fully proximally withdrawn into the needle enclosure 48, to prevent either the needle 30 being withdrawn inadvertently or prematurely, or the automatic needle withdrawal device 44 being disconnected from the catheter hub 12 inadvertently or prematurely. In other words, the trigger mechanism 52 may be configured to prevent false triggering of the needle withdrawal mechanism 50.

The needle withdrawal mechanism 50 may, for example, comprise a spring-loaded plunger 54 arranged inside the needle enclosure 48. The spring-loaded plunger 54 may be configured to mount to a proximal end of the needle 30, and may be spring-biased to proximally withdraw the needle 30 into the needle enclosure 48.

In the embodiment shown in FIGS. 5 to 8, the trigger mechanism 52 may, for example, comprise a leaf spring arm integrally formed on the distal end of the automatic needle withdrawal device 44. The leaf spring arm may be activated automatically upon withdrawal of the needle enclosure 48 from the catheter hub 12 to trigger automatic proximal withdrawal of the needle 30.

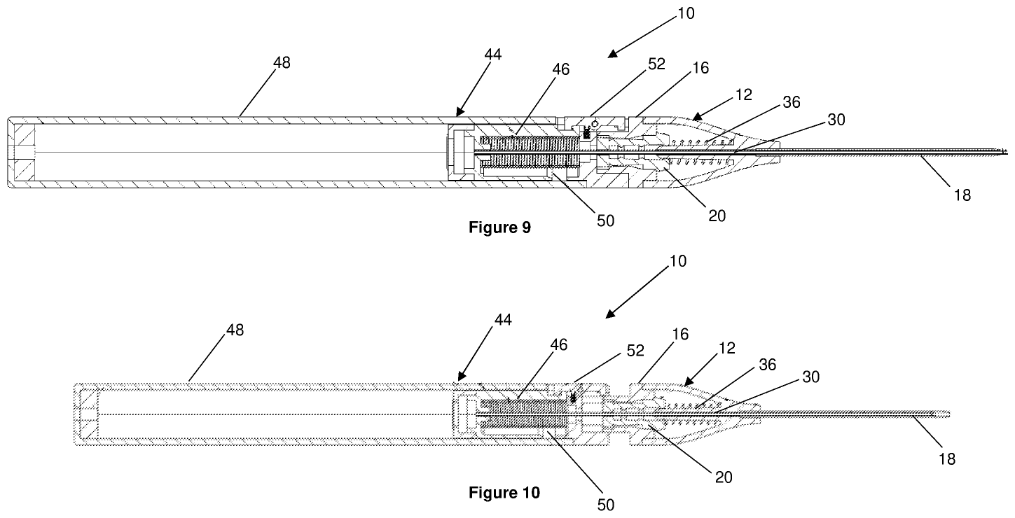

In the alternative embodiment shown in FIGS. 9 to 12, the trigger mechanism 52 may, for example, comprise a non-integrally formed spring-loaded reciprocating arm arranged between the distal end of the automatic needle withdrawal device 44 and the proximal end of the catheter hub 12. The spring-loaded reciprocating arm may be activated automatically upon withdrawal of the needle enclosure 48 from the catheter hub 12 to trigger automatic proximal withdrawal of the needle 30.

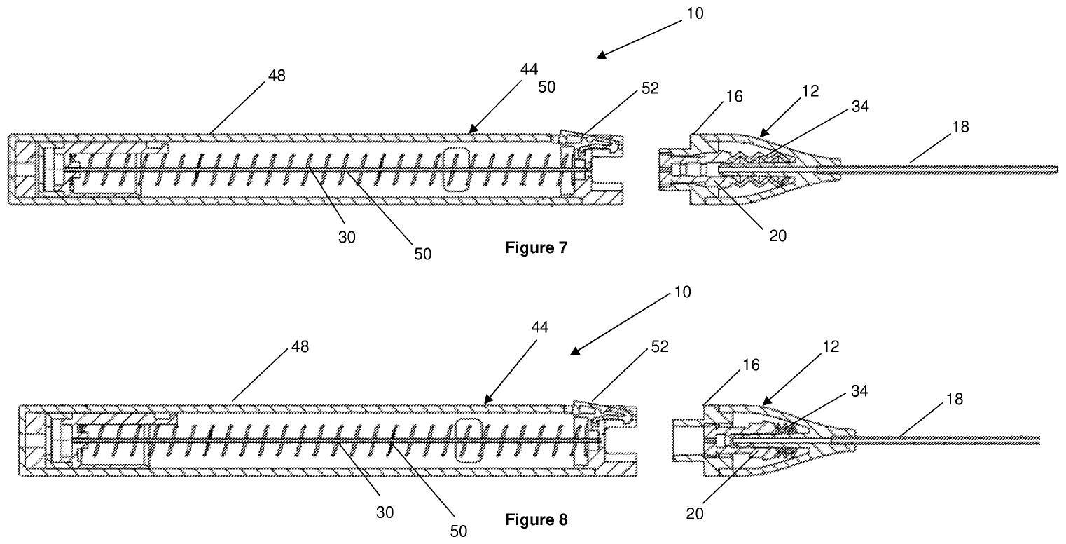

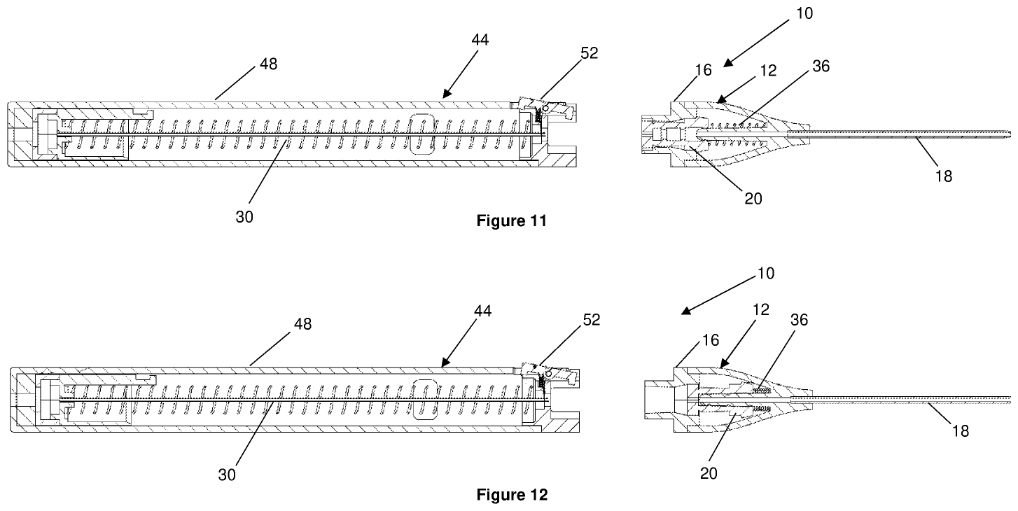

FIGS. 5 to 8 and 9 to 12 show alternative embodiments of the assembly 10 in each of four successive operational states that may be referred to, respectively, as:
a first state ‘ready-to-use’ for needle insertion (FIGS. 5 and 9);
a second state where the automatic needle withdrawal device 44 is ‘triggered’ with partial needle withdrawal (FIGS. 6 and 10);
a third state where the needle is fully withdrawn and enclosed (FIGS. 7 and 11); and
a fourth state ‘ready-to-use’ for intravenous fluid administration (FIGS. 8 and 12).

The components of the assembly 10 may generally be formed as mouldings of plastics material. The exceptions may be the pinch valve body 20 which may be formed of mouldings of elastomeric materials, or composites of plastics material and elastomeric materials. In addition, the needle 30 and springs 34 may be made of a metal or metal alloy. The plastics, elastomeric, composite, and metal materials may all be of suitable medical grade.

Suitable components of the assembly 10, such as the pinch valve body 20 and the catheter tube 18, may be made from or modified with suitable antimicrobial medical grade materials to avoid or minimise the need for aseptic wiping prior to interaction with a male luer connector, thereby avoiding or minimising risks of patient infection.

Embodiments of the present invention provide an over-the-needle intravenous introducer assembly that is both generally and specifically useful for introducing cannulas or catheters into veins of patients with improved adherence to aseptic technique and resulting reduced risk of patient infections, as well as reduced risks of needlestick injury and blood contamination to medical personnel.

Embodiments of the invention may facilitate increased adherence to aseptic non-touch technique and reduce the risks of needlestick injury and blood contamination without requiring additional components, steps and time to introduce the cannula or catheter into the patient's vein, thereby providing several advantages over existing over-the-needle introducer assemblies and addressing the shortcomings of existing over-the-needle introducer assemblies described above. This may be particularly advantageous in military and emergency medical settings, including pre-hospital (or battlefield) and in-hospital emergency medical settings.

Unless the context requires otherwise, the word “comprising” means “including but not limited to,” and the word “comprises” has a corresponding meaning.

Any reference to prior art is not an admission that the prior art is common general knowledge anywhere unless the context implies otherwise.

The scope of the invention supported by the above examples is defined by the claims that follow.

1. An assembly, comprising:
a catheter hub defining a hub cavity extending axially between proximal and distal ends of the catheter hub;
a female luer connector extending from within the proximal end of the catheter hub;
a catheter tube extending from within the distal end of the catheter hub; and
a pinch valve body axially movable in the hub cavity and defining a valve conduit that fluidly connects the female luer connector and the catheter tube;
wherein the pinch valve body has a distal end sheathed over the catheter tube, and a proximal end comprising proximal and distal pinch valves;
wherein the female luer connector has internal pinch surfaces configured so that axial movement of the pinch valve body in the hub cavity positively opens and closes the proximal and distal pinch valves in predetermined succession or order as either a needle is proximally withdrawn from the catheter tube, or a male luer connector is distally inserted into, or proximally withdrawn from, the female luer connector.

2. The assembly of claim 1, wherein the proximal and distal pinch valves comprise a luer receiving valve and an internal shut-off valve respectively.

3. The assembly of claim 1, wherein the proximal and distal pinch valves are closed initially, and a needle is pre-inserted in the catheter hub with a proximal portion in the valve conduit of the pinch valve body and a distal portion in the catheter tube.

4. The assembly of claim 3, wherein partial proximal withdrawal of the needle from the catheter hub opens the distal and proximal pinch valves in succession.

5. The assembly of claim 4, wherein full proximal withdrawal of the needle from the catheter hub closes the distal and proximal pinch valves in succession.

6. The assembly of claim 5, wherein distal insertion of the male luer connector into the female luer connector of the catheter hub opens the proximal and distal pinch valves in succession.

7. The assembly of claim 6, wherein proximal withdrawal of the male luer connector from the female luer connector of the catheter hub closes the distal and proximal pinch valves in succession.

8. The assembly of claim 1, wherein the pinch valve body is biased distally within the hub cavity to positively close the proximal and distal pinch valves.

9. The assembly of claim 8, wherein:
the catheter hub has an inwardly projecting tube portion at the distal end of the catheter hub;
the catheter tube extends distally from within the inwardly projecting tube portion; and
the pinch valve body is axially movable on the inwardly projecting tube portion.

10. The assembly of claim 9, wherein the pinch valve body is biased distally within the hub cavity by a spring sheathed on the inwardly projecting tube portion.

11. The assembly of claim 9, wherein elastomeric projections are provided at the distal end of the valve body, and wherein the elastomeric projections are sheathed over the inwardly projecting tube portion of the hub cavity to distally bias the pinch valve body.

12. The assembly of claim 1, further comprising a manual needle withdrawal device removably connected to the female luer connector of the catheter hub, wherein the manual needle withdrawal device has a low form factor and is configured so that disconnection from the catheter hub manually distally withdraws the needle from the catheter hub and automatically applies a safety clip to the distal tip of the needle when fully distally withdrawn from the catheter hub.

13. The assembly of claim 12, wherein the manual needle withdrawal device comprises a flashback chamber configured to allow visualisation of an initial flow of blood upon insertion of a distal tip of the needle into a vein.

14. The assembly of claim 1, further comprising an automatic needle withdrawal device removably connected to the female luer connector of the catheter hub, wherein the automatic needle withdrawal device comprises:
a flashback chamber configured to allow visualisation of an initial flow of blood upon insertion of a distal tip of the needle into a vein;
a needle enclosure configured to enclose the needle when fully distally withdrawn from the catheter hub;
a needle withdrawal mechanism configured to be triggered to automatically distally withdraw the needle from the catheter hub and move the needle to the needle enclosure when fully distally withdrawn from the catheter hub; and
a trigger mechanism configured to:
trigger the needle withdrawal mechanism to fully proximally withdraw the needle into the needle enclosure, and disconnect the automatic needle withdrawal device from the catheter hub after the needle is fully proximally withdrawn into the needle enclosure; and
before the needle is fully proximally withdrawn into the needle enclosure, prevent either:
the needle being withdrawn inadvertently or prematurely; or
the automatic needle withdrawal device being disconnected from the catheter hub inadvertently or prematurely.

15. The assembly of claim 14, wherein the needle withdrawal mechanism comprises a spring-loaded plunger arranged inside the needle enclosure, wherein the spring-loaded plunger is configured to mount to a proximal end of the needle and is spring-biased to proximally withdraw the needle into the needle enclosure.

16. The assembly of claim 14, wherein the trigger mechanism comprises a spring arm integrally formed on a distal end of the automatic needle withdrawal device, and wherein the leaf spring arm is configured to be activated automatically upon proximal withdrawal of the needle enclosure from the catheter hub to trigger automatic proximal withdrawal of the needle.

17. The assembly of claim 14, wherein the trigger mechanism comprises a non-integrally formed spring-loaded reciprocating arm arranged between a distal end of the automatic needle withdrawal device and the proximal end of the catheter hub, wherein the spring-loaded reciprocating arm is configured to be activated automatically upon proximal withdrawal of the needle enclosure from the catheter hub to trigger automatic proximal withdrawal of the needle.

18. A method, comprising:
providing a needle inside a catheter tube at a distal end of a catheter hub having a female luer connector at a proximal end, wherein the catheter tube and female luer connector are fluidly connected by a pinch valve body that is proximally movable inside a hub cavity of the catheter hub, and wherein the pinch valve body has a distal end sheathed over the catheter tube, and a proximal end comprising proximal and distal pinch valves that are configured to be closed in succession by internal pinch surfaces of the female catheter connector by proximal movement of the pinch valve body;
proximally withdrawing the needle from the catheter tube to proximally move the pinch valve body;
closing the proximal and distal pinch valves in succession upon full proximal withdrawal of the needle from the catheter hub.
